(19) United States
(12) Patent Application Publication
(10) Pub. No.: US 2017/0165005 A1
(43) Pub. Date: Jun. 15, 2017

(54) PORT TRACKING TOOL

(71) Applicants: Leila KHERADPIR, Toronto (CA); William LAU, Toronto (CA); Gal SELA, Toronto (CA); Joshua Lee RICHMOND, Toronto (CA)

(72) Inventors: Leila KHERADPIR, Toronto (CA); William LAU, Toronto (CA); Gal SELA, Toronto (CA); Joshua Lee RICHMOND, Toronto (CA)

(21) Appl. No.: 15/323,760

(22) PCT Filed: Aug. 28, 2014
(86) PCT No.: PCT/CA2014/050822

§ 371 Date: Jan. 4, 2017

(51) Int. Cl. A61B 34/20; A61B 46/20; A61B 17/34; A61B 34/00; A61B 34/30

(52) U.S. Cl. CPC A61B 34/20; A61B 34/25; A61B 34/30; A61B 17/3423; A61B 46/20; A61B 2034/2055; A61B 2017/00902

(57) ABSTRACT

An access port tracking apparatus is provided having a frame including two arms positioned at a relative angle, a coupling member attached to the frame, the coupling member for coupling the tracking apparatus to an access port, and a coupling attached to the frame for connecting a tracking marker to the frame.

### TECHNICAL FIELD

The present disclosure is generally related to image guided medical procedures, and more specifically to a port tracking tool.

### BACKGROUND

The present disclosure is generally related to image guided medical procedures using a surgical instrument, such as a fiber optic scope, an optical coherence tomography (OCT) probe, a micro ultrasound transducer, an electronic sensor or stimulator, or an access port based surgery.

In the example of a port-based surgery, a surgeon or robotic surgical system may perform a surgical procedure involving tumor resection in which the residual tumor remaining after is minimized, while also minimizing the trauma to the intact white and grey matter of the brain. In such procedures, trauma may occur, for example, due to contact with the access port, stress to the brain matter, unintentional impact with surgical devices, and/or accidental resection of healthy tissue. A key to minimizing trauma is ensuring that the spatial reference of the patient as understood by the surgical system is as accurate as possible.

FIG. 1 illustrates the insertion of an access port into a human brain, for providing access to internal brain tissue during a medical procedure. In FIG. 1, access port 12 is inserted into a human brain 10, providing access to internal brain tissue. Access port 12 may include such instruments as catheters, surgical probes, or cylindrical ports such as the NICO BrainPath. Surgical tools and instruments may then be inserted within the lumen of the access port in order to perform surgical, diagnostic or therapeutic procedures, such as resecting tumors as necessary. The present disclosure applies equally well to catheters, DBS needles, a biopsy procedure, and also to biopsies and/or catheters in other medical procedures performed on other parts of the body.

In the example of a port-based surgery, a straight or linear access port 12 is typically guided down a sulci path of the brain. Surgical instruments would then be inserted down the access port 12.

Optical tracking systems, used in the medical procedure, track the position of a part of the instrument that is within line-of-site of the optical tracking camera. These optical tracking systems also require a reference to the patient to know where the instrument is relative to the target (e.g., a tumor) of the medical procedure. These optical tracking systems require a knowledge of the dimensions of the instrument being tracked so that, for example, the optical tracking system knows the position in space of a tip of a medical instrument relative to the tracking markers being tracked.

Conventional systems have shortcomings with respect to access port positioning because, once an access port is positioned in a patient during a procedure, the position of the access port is typically not subsequently tracked during the procedure. Therefore, there is a need for an improved approach for access port positioning during a medical procedure.

### SUMMARY

One aspect of the present disclosure provides an access port tracking apparatus comprising a frame, a coupling member attached to the frame, the coupling member for coupling the tracking apparatus to an access port, and a coupling attached to the frame for connecting a tracking marker to the frame. The access port may be substantially cylindrical having an outside circumference and the coupling member may be ring shaped for engaging the access port outside circumference. The coupling member may have a hole in the center with an inside circumference being substantially equal to the outside circumference of access port. The ring shaped coupling member may further include a plurality of locking members formed on an upper surface of the coupling member for engaging an underside of a lip located around the outside circumference of the access port near a top of the access port. The frame may include two substantially linear arms positioned at a relative angle with between 110 degrees and 130 degrees between the two arms, each of the two arms including two tracking markers attached thereto. A tracking marker may be attached to the coupling. The coupling includes a threaded stud and the tracking marker has a threaded hole such that the tracking marker is screwed onto the threaded stud.

Another aspect of the present disclosure provides a medical navigation system having an access port, an access port tracking apparatus, and a controller. The access port tracking apparatus has a frame, a coupling member attached to the frame, the coupling member for coupling the tracking apparatus to the access port, and a coupling attached to the frame for connecting a tracking marker to the frame. The controller is at least electrically coupled to a sensor, the sensor providing a signal to the controller indicating movement of the tracking marker.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the insertion of an access port into a human brain, for providing access to internal brain tissue during a medical procedure;

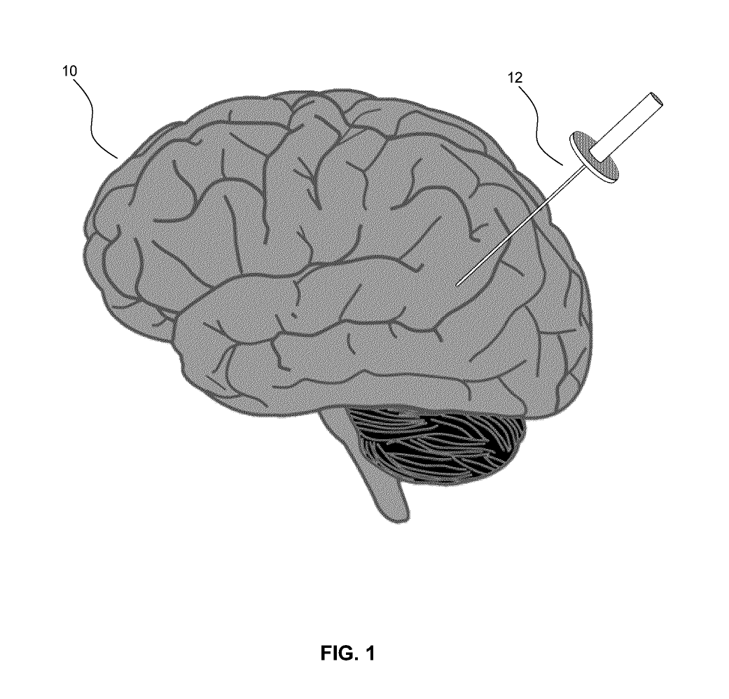

FIG. 2 shows an exemplary navigation system to support minimally invasive access port-based surgery;

FIG. 3 is a block diagram illustrating a control and processing system that may be used in the navigation system shown in FIG. 2;

FIG. 4A is a flow chart illustrating a method involved in a surgical procedure using the navigation system of FIG. 2;

FIG. 4B is a flow chart illustrating a method of registering a patient for a surgical procedure as outlined in FIG. 4A;

FIG. 5A is a perspective drawing illustrating an exemplary context for aspects of the present application including an access port, port tracking tool, and medical tool;

FIG. 5B is an exploded view of the drawing shown in FIG. 5A;

FIG. 6 is a perspective drawing illustrating in isolation the exemplary port tracking tool and access port introduced in FIG. 5;

FIG. 7 is a front view of the port tracking tool and access port shown in FIG. 6;

FIG. 8 is a right side view of the port tracking tool and access port shown in FIG. 6; and

FIG. 9 is a rear view of the port tracking tool and access port shown in FIG. 6.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

Unless defined otherwise, all technical and scientific terms used herein are intended to have the same meaning as commonly understood by one of ordinary skill in the art. Unless otherwise indicated, such as through context, as used herein, the following terms are intended to have the following meanings:

As used herein, the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof. Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port.

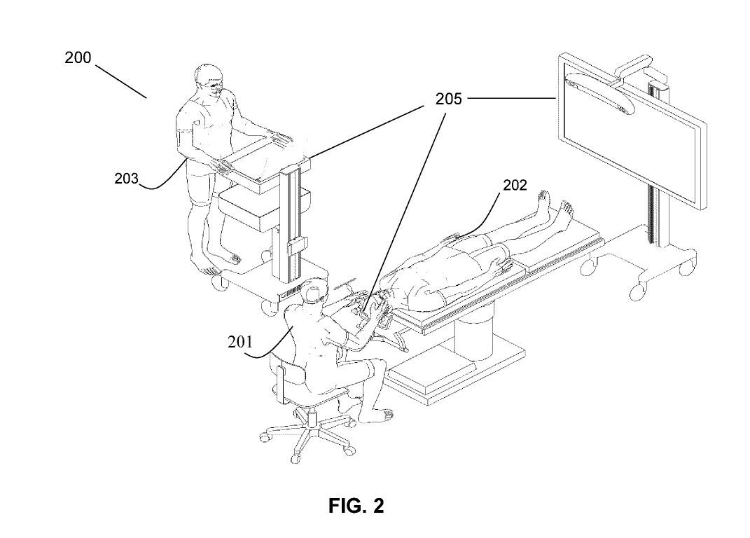

Referring to FIG. 2, an exemplary navigation system environment 200 is shown, which may be used to support navigated image-guided surgery. As shown in FIG. 2, surgeon 201 conducts a surgery on a patient 202 in an operating room (OR) environment. A medical navigation system 205 comprising an equipment tower, tracking system, displays and tracked instruments assist the surgeon 201 during his procedure. An operator 203 is also present to operate, control and provide assistance for the medical navigation system 205.

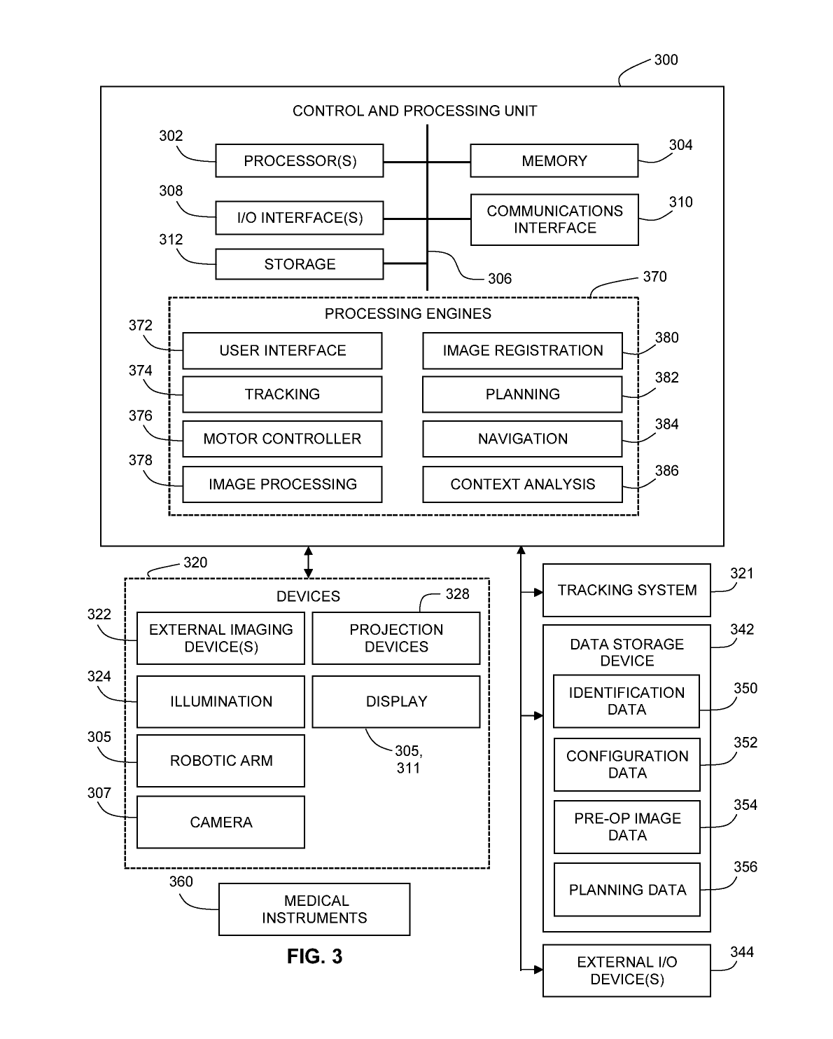

Referring to FIG. 3, a block diagram is shown illustrating a control and processing system 300 that may be used in the medical navigation system 200 shown in FIG. 3 (e.g., as part of the equipment tower). As shown in FIG. 3, in one example, control and processing system 300 may include one or more processors 302, a memory 304, a system bus 306, one or more input/output interfaces 308, a communications interface 310, and storage device 312. Control and processing system 300 may be interfaced with other external devices, such as tracking system 321, data storage 342, and external user input and output devices 344, which may include, for example, one or more of a display, keyboard, mouse, sensors attached to medical equipment, foot pedal, and microphone and speaker. Data storage 342 may be any suitable data storage device, such as a local or remote computing device (e.g. a computer, hard drive, digital media device, or server) having a database stored thereon. In the example shown in FIG. 3, data storage device 342 includes identification data 350 for identifying one or more medical instruments 360 and configuration data 352 that associates customized configuration parameters with one or more medical instruments 360. Data storage device 342 may also include preoperative image data 354 and/or medical procedure planning data 356. Although data storage device 342 is shown as a single device in FIG. 3, it will be understood that in other embodiments, data storage device 342 may be provided as multiple storage devices.

Medical instruments 360 are identifiable by control and processing unit 300. Medical instruments 360 may be connected to and controlled by control and processing unit 300, or medical instruments 360 may be operated or otherwise employed independent of control and processing unit 300. Tracking system 321 may be employed to track one or more of medical instruments 360 and spatially register the one or more tracked medical instruments to an intraoperative reference frame. For example, medical instruments 360 may include tracking markers such as tracking spheres that may be recognizable by a tracking camera 307. In one example, the tracking camera 307 may be an infrared (IR) tracking camera. In another example, as sheath placed over a medical instrument 360 may be connected to and controlled by control and processing unit 300.

Control and processing unit 300 may also interface with a number of configurable devices, and may intraoperatively reconfigure one or more of such devices based on configuration parameters obtained from configuration data 352. Examples of devices 320, as shown in FIG. 3, include one or more external imaging devices 322, one or more illumination devices 324, a robotic arm, one or more projection devices 328, and one or more displays 205, 211.

Exemplary aspects of the disclosure can be implemented via processor(s) 302 and/or memory 304. For example, the functionalities described herein can be partially implemented via hardware logic in processor 302 and partially using the instructions stored in memory 304, as one or more processing modules or engines 370. Example processing modules include, but are not limited to, user interface engine 372, tracking module 374, motor controller 376, image processing engine 378, image registration engine 380, procedure planning engine 382, navigation engine 384, and context analysis module 386. While the example processing modules are shown separately in FIG. 3, in one example the processing modules 370 may be stored in the memory 304 and the processing modules may be collectively referred to as processing modules 370.

It is to be understood that the system is not intended to be limited to the components shown in FIG. 3. One or more components of the control and processing system 300 may be provided as an external component or device. In one example, navigation module 384 may be provided as an external navigation system that is integrated with control and processing system 300.

Some embodiments may be implemented using processor 302 without additional instructions stored in memory 304. Some embodiments may be implemented using the instructions stored in memory 304 for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which, when executed by a data processing system, causes the system to perform various methods. The executable software and data may be stored in various places including for example ROM, volatile RAM, nonvolatile memory and/or cache. Portions of this software and/or data may be stored in any one of these storage devices.

Examples of computer-readable storage media include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions may be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

At least some of the methods described herein are capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

According to one aspect of the present application, one purpose of the navigation system 205, which may include control and processing unit 300, is to provide tools to the neurosurgeon that will lead to the most informed, least damaging neurosurgical operations. In addition to removal of brain tumours and intracranial hemorrhages (ICH), the navigation system 205 can also be applied to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

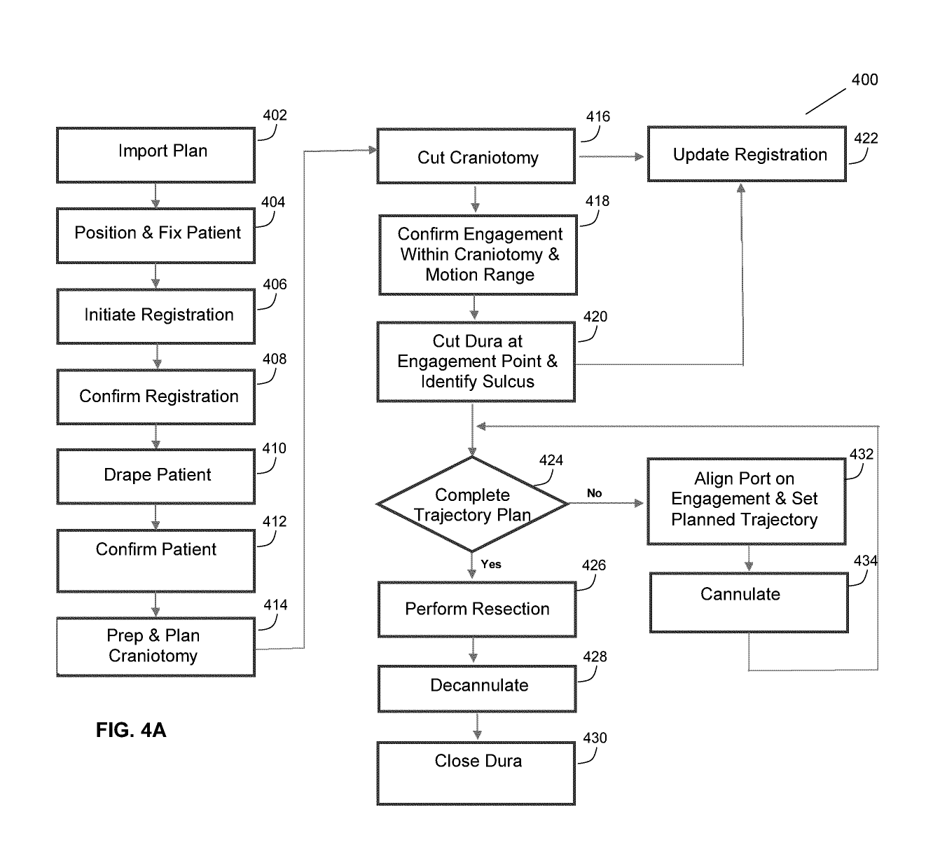

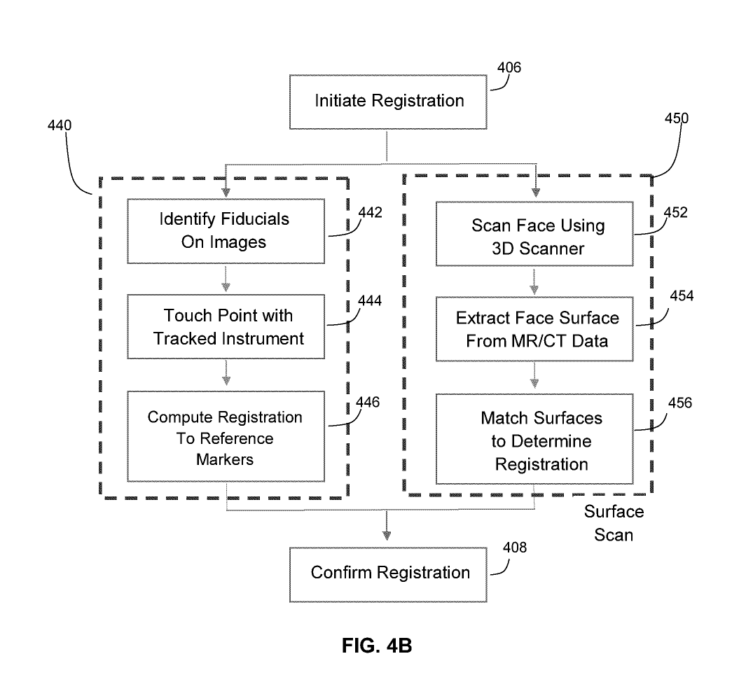

Referring to FIG. 4A, a flow chart is shown illustrating a method 400 of performing a port-based surgical procedure using a navigation system, such as the medical navigation system 200 described in relation to FIG. 2. At a first block 402, the port-based surgical plan is imported. A detailed description of the process to create and select a surgical plan is outlined in the disclosure “PLANNING, NAVIGATION AND SIMULATION SYSTEMS AND METHODS FOR MINIMALLY INVASIVE THERAPY”, a United States Patent Publication based on a United States Patent Application, which claims priority to U.S. Provisional Patent Application Ser. Nos. 61/800,155 and 61/924,993, which are both hereby incorporated by reference in their entirety.

Once the plan has been imported into the navigation system at the block 402, the patient is affixed into position using a body holding mechanism. The head position is also confirmed with the patient plan in the navigation system (block 404), which in one example may be implemented by the computer or controller forming part of the equipment tower 201.

Next, registration of the patient is initiated (block 406). The phrase “registration” or “image registration” refers to the process of transforming different sets of data into one coordinate system. Data may include multiple photographs, data from different sensors, times, depths, or viewpoints. The process of “registration” is used in the present application for medical imaging in which images from different imaging modalities are co-registered. Registration is used in order to be able to compare or integrate the data obtained from these different modalities.

Those skilled in the relevant arts will appreciate that there are numerous registration techniques available and one or more of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT.

Referring now to FIG. 4B, a flow chart is shown illustrating a method involved in registration block 406 as outlined in FIG. 4A, in greater detail. If the use of fiducial touch points (440) is contemplated, the method involves first identifying fiducials on images (block 442), then touching the touch points with a tracked instrument (block 444). Next, the navigation system computes the registration to reference markers (block 446).

Alternately, registration can also be completed by conducting a surface scan procedure (block 450). The block 450 is presented to show an alternative approach, but may not typically be used when using a fiducial pointer. First, the face is scanned using a 3D scanner (block 452). Next, the face surface is extracted from MR/CT data (block 454). Finally, surfaces are matched to determine registration data points (block 456).

Upon completion of either the fiducial touch points (440) or surface scan (450) procedures, the data extracted is computed and used to confirm registration at block 408, shown in FIG. 4A.

Referring back to FIG. 4A, once registration is confirmed (block 408), the patient is draped (block 410). Typically, draping involves covering the patient and surrounding areas with a sterile barrier to create and maintain a sterile field during the surgical procedure. The purpose of draping is to eliminate the passage of microorganisms (e.g., bacteria) between non-sterile and sterile areas. At this point, conventional navigation systems require that the non-sterile patient reference is replaced with a sterile patient reference of identical geometry location and orientation. Numerous mechanical methods may be used to minimize the displacement of the new sterile patient reference relative to the non-sterile one that was used for registration but it is inevitable that some error will exist. This error directly translates into registration error between the surgical field and pre-surgical images. In fact, the further away points of interest are from the patient reference, the worse the error will be.

Upon completion of draping (block 410), the patient engagement points are confirmed (block 412) and then the craniotomy is prepared and planned (block 414).

Upon completion of the preparation and planning of the craniotomy (block 414), the craniotomy is cut and a bone flap is temporarily removed from the skull to access the brain (block 416). Registration data is updated with the navigation system at this point (block 422).

Next, the engagement within craniotomy and the motion range are confirmed (block 418). Next, the procedure advances to cutting the dura at the engagement points and identifying the sulcus (block 420).

Thereafter, the cannulation process is initiated (block 424). Cannulation involves inserting a port into the brain, typically along a sulci path as identified at 420, along a trajectory plan. Cannulation is typically an iterative process that involves repeating the steps of aligning the port on engagement and setting the planned trajectory (block 432) and then cannulating to the target depth (block 434) until the complete trajectory plan is executed (block 424).

Once cannulation is complete, the surgeon then performs resection (block 426) to remove part of the brain and/or tumor of interest. The surgeon then decannulates (block 428) by removing the port and any tracking instruments from the brain. Finally, the surgeon closes the dura and completes the craniotomy (block 430). Some aspects of FIG. 4A are specific to port-based surgery, such as portions of blocks 428, 420, and 434, but the appropriate portions of these blocks may be skipped or suitably modified when performing non-port based surgery.

When performing a surgical procedure using a medical navigation system 200, as outlined in connection with FIGS. 4A and 4B, the medical navigation system 200 must acquire and maintain a reference of the location of the tools in use as well as the patient in three dimensional (3D) space. In other words, during a navigated neurosurgery, there needs to be a tracked reference frame that is fixed relative to the patient's skull. During the registration phase of a navigated neurosurgery (e.g., the step 406 shown in FIGS. 4A and 4B), a transformation is calculated that maps the frame of reference of preoperative MRI or CT imagery to the physical space of the surgery, specifically the patient's head. This may be accomplished by the navigation system 200 tracking locations of fiducial markers fixed to the patient's head, relative to the static patient reference frame. The patient reference frame is typically rigidly attached to the head fixation device, such as a Mayfield clamp. Registration is typically performed before the sterile field has been established (e.g., the step 410 shown in FIG. 4A).

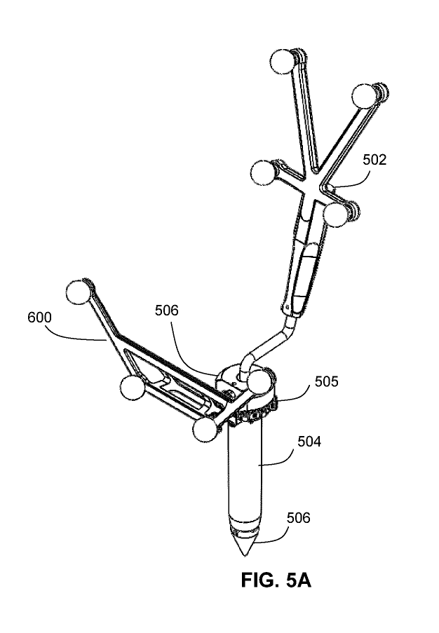

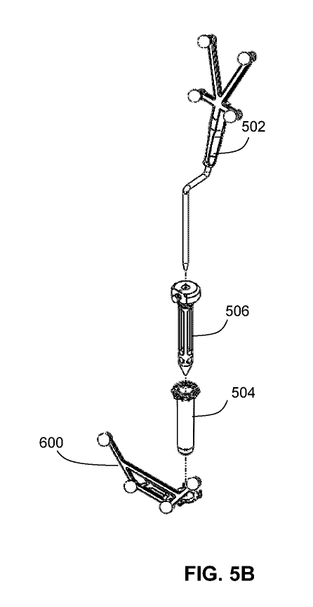

Referring to FIG. 5A, a perspective drawing is shown illustrating an exemplary context for aspects of the present application including a medical tool 502, an access port 504, an obturator 506, and a port tracking tool 600. FIG. 5B is an exploded view of the drawing shown in FIG. 5A. FIGS. 5A and 5B will be collectively referred to as FIG. 5 and are now discussed concurrently. Port-based neurosurgery is a minimally-invasive procedure. Currently, a navigation system such as the medical navigation system 205 using the control and processing unit 300 is used to track a pointer tool, such as the medical tool 502, inserted into the obturator 506 of the port sheath (e.g., the access port 504) during the approach phase of the surgery. Navigation in approach facilitates placement of the sheath or access port in the correct location close to the target area of the brain along a planned trajectory. When the navigation system 205 is used in conventional approaches, the pointer tool (e.g., the medical tool 502) is introduced into the sheath or port momentarily to orient the surgeon relative to preoperative Magnetic Resonance (MR) or Computed Tomography (CT) images.

There are at least two opportunities to solve problems by tracking the access port 504 continuously. First, in approach, the final step is to decant the sheath or access port 504 by moving the sheath or access port 504 down to the tip of the obturator 506. Often, surgeons who are new to the procedure will retract the obturator 506 instead of moving the access port 504 down. Since the access port 504 is not tracked, it is not clear from the medical navigation system display (e.g., the display 305, 311) that the access port 504 ended up in the wrong location. Second, during resection, real-time tracking of the access port 504 would provide the surgeon with a continuous view of where he is operating (e.g., per preoperative images). The use of a tracked access port 504 would also reduce the need for the surgeon to put down his surgical tool(s) in order to reintroduce the navigated pointer tool 502 down the access port 504. Yet another possible benefit is that if the sheath or access port 504 is displaced along the length of the obturator 506 during approach, tracking the access port 504 continuously allows for detection and display of the displacement to the surgeon.

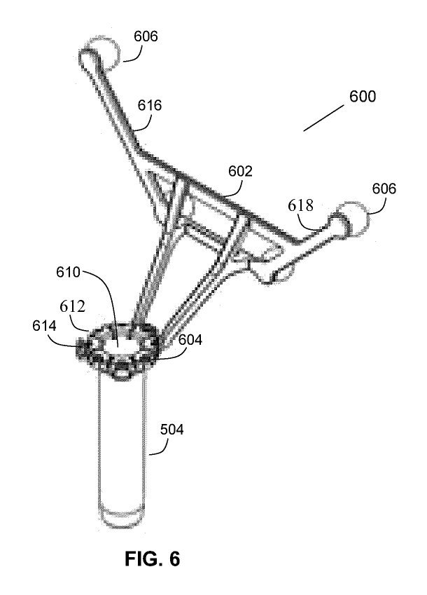

The problems with the conventional approach can be solved or reduced by continuously tracking the location of the access port 504 during a medical procedure. This may be achieved by using the port tracking tool 600, discuss in more detail below in connection with FIGS. 6-9.

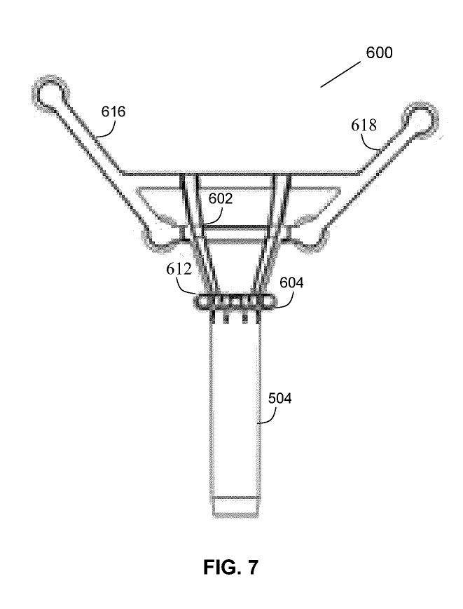

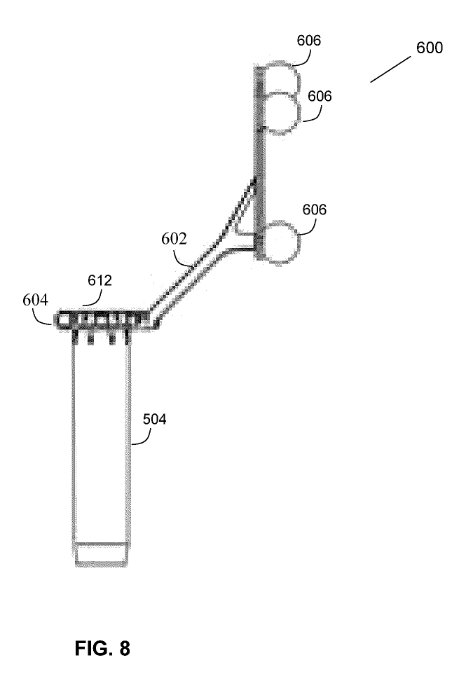

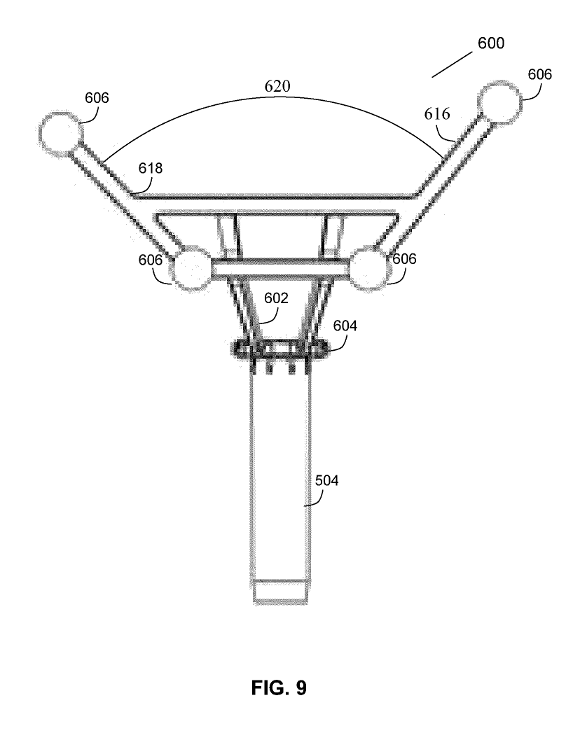

Referring now to FIG. 6, a perspective drawing is shown illustrating in isolation the exemplary port tracking tool 600 attached to access port 504. FIG. 7 is a front view of the port tracking tool 600 attached to access port 504, shown in FIG. 6. FIG. 8 is a right side view of the port tracking tool 600 attached to access port 504, shown in FIG. 6. FIG. 9 is a rear view of the port tracking tool attached to access port 504, shown in FIG. 6. FIGS. 6-9 will now be discussed concurrently.

The access port tracking tool 600 is referred to interchangeably as either the access port tracking tool 600 or the access port tracking apparatus 600. The access port tracking apparatus 600 includes a frame 602 and a coupling member 604 attached to the frame. The coupling member 604 couples the access port tracking apparatus 600 to the access port 504. At least one coupling is attached to the frame (not shown) for connecting a tracking marker 606 to the frame 602. In another example, at least three tracking markers 606 are attached to at least three couplings of the frame 602. In one example, the coupling may be a threaded stud and the tracking marker 606 may have a threaded hole for screwing the tracking marker 606 onto the threaded stud. In another example, the stud and the hold may be without a thread and the tracking marker 606 may be press fit onto the coupling. While two examples of attaching the tracking markers 606 to the couplings have been provided, the tracking markers 606 may be attached to couplings on the frame 602 using any suitable mechanism.

In one example, the access port 504 may be substantially cylindrical and have an outside circumference and the coupling member 604 may be ring shaped for engaging the access port 504 outside circumference. The coupling member having a hole in the center, indicated by reference 610, with an inside circumference being approximately or substantially equal to the outside circumference of access port 504. The coupling member 604 may further include a plurality of locking members 612 formed on an upper surface of the coupling member 604 for engaging an underside of a lip 505 (FIG. 5) located around the outside circumference of the access port 504 near a top of the access port. In one example, the coupling member 604 may further include a number of recesses 614 around the outside circumference of the coupling member 604. In one example, the recesses 614 may be used by a surgeon to clock the sheath or access port 504 while rotating the access port 504 in the surgical site. The locking members 612 and the recesses 614 may be optional features and in some examples the access port 504 may simply be friction fit to the coupling member 604.

The tracking marker 606 used for the port tracking tool 600 may include any of a passive reflective tracking sphere, an active infrared (IR) marker, an active light emitting diode (LEDs), or a graphical pattern. In the example shown in FIGS. 6-9, passive reflecting tracking spheres may be used. Typically at least three passive reflective tracking spheres may be used. In the example shown in FIGS. 6-9, four passive reflective tracking spheres may be used and may be attached to the frame 602. The example shown in FIGS. 6-9 shows four specific tracking marker locations 606, however tracking makers 606 may be located anywhere on frame 602 and frame 602 may have any suitable shape for supporting the tracking markers 606 according to the design criteria of a particular application.

In the example shown in FIGS. 6-9, a plane is defined by tops of the passive reflective tracking markers 606. Alternatively, it may be said that the tops of the tracking markers 606 (e.g., shown best in FIG. 8) may define a plane. This plane defined by the tops of the passive reflective tracking makers 606 may be substantially perpendicular to an insertion plane of the access port (e.g., a plane that is normal to the axis of the access port 504). In one example, the passive reflective tracking makers 606 may be located at least 20 mm above the access port 504 when the access port 504 is coupled to the access port tracking apparatus 600 to allow for easy viewing of the tracking makers 606 by a tracking camera (e.g., the camera 307 and/or the tracking system 321) coupled to a medical navigation system 205. This physical relationship of the tracking markers 606 relative to the access port 504 is exemplary only, and any suitable physical relationship may be used according to the design criteria of a particular application.

In one example, the access port tracking apparatus 600 may be constructed from a lightweight polymer. In one example, the lightweight polymer may be biocompatible and sterilizable. In one example, the lightweight polymer may be anyone of liquid crystal polymer (LCP), polycarbonate, polyether ether ketone (PEEK), Ultem™, polytetrafluoroethylene (PTFE), or Acetel. In one example, the lightweight polymer may be LCP-TRP 3405-3. While some examples of suitable polymers have been provided, the access port tracking apparatus 600 may be constructed of any suitable existing or yet to be developed lightweight polymer. In another example, the access port tracking apparatus 600 may be constructed from a lightweight metal. Whether constructed from a polymer or from metal, the frame 602 may be stiff enough to resist bending or deformation under normal use.

In one example, the frame 602 includes two substantially linear arms 616 and 618. In one example, the arms 616, 618 may be positioned at a relative angle 620 (FIG. 9) with between 110 degrees and 130 degrees between the two arms 616, 618, each of the two arms 616, 618 including two tracking marker mounting locations for tracking makers 606 (best shown in FIG. 9). In one example, the angle 620 may be such that arms 616, 618 are positioned at approximately 120 degrees relative to each other, which may provide an optimum or nearly optimum configuration to avoid interfering with the field of view of a surgeon using the port tracking tool 600 and to avoid interfering with access for the surgeon's hands and surgical tools to the surgical site. In other words, the port tracking tool 600 may not impinge on the radial space from 60 degrees to 300 degrees with respect to the direction the surgeon is facing. The location of the two arms 616, 618 may be based on their relative position to the tracking camera (e.g., camera 307 and/or tracking system 321) for full visibility while giving the surgeon ample space for working the scope and surgical instruments. While an exemplary range of 110 degrees to 130 degrees for the relative angle 620 is provided, any suitable angle may be used to meet the design criteria of a particular application.

The two arms 616, 618 are attached to the coupling member 604 by the remainder of the frame 602 and the two arms 616, 618 may be spaced away from the coupling member 604 as shown in FIGS. 6-9.

The exemplary port tracking tool 600 shown in FIGS. 6-9 may be designed to interface with a NICO BrainPath Kit. The port tracking tool 600 may be suitably modified to interface with any known or yet to be developed access port, such as the access port 504.

The port tracking tool 600 allows the medical navigation system 205 to track the access port 504 throughout the course of a medical procedure. In one example, the port tracking tool 600 may be disposable and pre-sterilized and may be manufactured using a light weight polymer that is molded and, if necessary, machined. In one example, the port tracking tool 600 may be biocompatible as a limited exposure externally communicating device in direct contact with tissue, bone, or dentin and comply with the standard defined in ISO 10993 that is typically followed for evaluation. In one example, the port tracking tool 600 may be provided as a sterile device in accordance with applicable standards. In one example, the port tracking tool 600 may be compatible with rings of the following NICO Neuro BrainPath® devices: 60 mm length, 50 mm length (15 mm and 8 mm tip), and 75 mm length. However, the port tracking tool 600 may be suitably modified to interface with any known or yet to be developed access port. In another example, the port tracking tool 600 may not detach from the access port 504 under normal tool use (e.g., as achieved by the locking members 612 interfacing with the access port 504) and the port tracking tool 600 may be able to repeatedly attach to the access port 504 with a minimum repeatable desired accuracy according to the design criteria of a particular application.

While a separate access port 504 and port tracking apparatus 600 have been described, in some examples the access port 504 and port tracking apparatus 600 may be one integrated unit such that the access port 504 and port tracking apparatus 600 are formed at the same time using a suitable lightweight polymer or metal creating a single unit.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

We claim:

1. An access port tracking apparatus (600) comprising:
a frame (602) including two arms positioned at a relative angle;
a coupling member (604) attached to the frame, the coupling member for coupling the tracking apparatus to an access port; and
a coupling attached to the frame for connecting a tracking marker (606) to the frame.

2. The access port tracking apparatus (600) according to claim 1, wherein the apparatus (600) further comprises:
a tracking marker (606) attached to the coupling.

3. The access port tracking apparatus (600) according to any one of claims 1 and 2, wherein the access port is substantially cylindrical having an outside circumference and the coupling member is ring shaped for engaging the access port outside circumference.

4. The access port tracking apparatus (600) according to any one of claims 1-3, wherein the coupling includes a threaded stud and the tracking marker (606) has a threaded hole such that the tracking marker (606) is screwed onto the threaded stud.

5. The access port tracking apparatus (600) according to claim 3, the coupling member having a hole in the center with an inside circumference being substantially equal to the outside circumference of the access port.

6. The access port tracking apparatus (600) according to claim 5, wherein the ring shaped coupling member further includes a plurality of locking members formed on an upper surface of the coupling member for engaging an underside of a lip located around the outside circumference of the access port near a top of the access port.

7. The access port tracking apparatus (600) according to claim 6, the ring shaped coupling member further including a number of recesses around an outside circumference of the coupling member.

8. The access port tracking apparatus (600) according to any one of claims 1-7, wherein the tracking marker (606) is selected from the group consisting of passive reflective tracking spheres, active infrared (IR) markers, active light emitting diodes (LEDs), and a graphical pattern.

9. The access port tracking apparatus (600) according to any one of claims 1-8, wherein the access port tracking apparatus has at least three passive reflective tracking makers attached to at least three respective couplings on the frame.

10. The access port tracking apparatus (600) according to claim 9, wherein a plane defined by tops of the passive reflective tracking makers is substantially perpendicular to an insertion plane of the access port.

11. The access port tracking apparatus (600) according to claim 9, wherein the passive reflective tracking makers are located at least 20 mm above the access port when the access port is coupled to the access port tracking apparatus to allow for easy viewing of the tracking makers by a tracking camera coupled to a medical navigation system.

12. The access port tracking apparatus (600) according to any one of claims 1-11, wherein the access port tracking apparatus is constructed from one of a lightweight polymer and a lightweight metal.

13. The access port tracking apparatus (600) according to any one of claims 1-12, wherein the two arms are substantially linear and the relative angle is between 110 degrees and 130 degrees between the two substantially linear arms, each of the two arms including two tracking markers attached thereto.

14. The access port tracking apparatus (600) according to claim 13, wherein the two substantially linear arms are attached to the coupling member by the remainder of the frame and the two substantially linear arms are spaced away from the coupling member.

15. A medical navigation system (200), comprising:
an access port (504);
an access port tracking apparatus (600) having:
a frame (602);
a coupling member (604) attached to the frame, the coupling member for coupling the tracking apparatus to the access port; and
a coupling attached to the frame for connecting a tracking marker (606) to the frame;
a controller (300) at least electrically coupled to a sensor, the sensor providing a signal to the controller indicating movement of a tracking marker.

16. The medical navigation system (200) according to claim 15, wherein the sensor includes an optical tracking camera.

17. The medical navigation system (200) according to claim 15, wherein the system (200) further comprises:
a tracking marker (606) attached to the coupling.

18. The medical navigation system (200) according to any one of claims 15-17, wherein the coupling includes a threaded stud and the tracking marker (606) has a threaded hole such that the tracking marker (606) is screwed onto the threaded stud.
